(19) United States
(12) Patent Application Publication
(10) Pub. No.: US 2021/0121834 A1
(43) Pub. Date: Apr. 29, 2021

(54) CONCENTRATE AND DILUTION CARTRIDGE AND SYSTEMS

(71) Applicant: Applied Silver, Inc., Hayward, CA (US)

(72) Inventors: Austin Carey, Westlake Village, CA (US); Priya Balachandran, Palo Alto, CA (US); Elizabeth Hutt Pollard, Edmond, OK (US); Sean Morham, San Francisco, CA (US)

(21) Appl. No.: 17/257,188

(22) PCT Filed: Jul. 5, 2019
(86) PCT No.: PCT/US2019/040699

§ 371 Date: Dec. 30, 2020

Related U.S. Application Data
(60) Provisional application No. 62/694,992, filed on Jul. 7, 2018.

(51) Int. Cl. B01F 3/08; G06K 7/10; B01F 15/04; A61L 2/18

(52) U.S. Cl. CPC B01F 3/0803; A61L 2/18; B01F 15/04; G06K 7/10009

(57) ABSTRACT

Cartridges and systems for delivering solutions to textiles. In various aspects, the cartridge may include (i) a concentrate chamber that stores a concentrate solution and comprising an outlet, (ii) a dilution chamber comprising an inlet for receiving the concentrate solution from the concentrate chamber, and outlet for dispensing a diluted solution, and (iii) a mechanical portion comprising one or more pumps for delivering the concentrate solution to the dilution chamber and for dispensing the diluted solution. Delivery systems using the cartridge are described.

### CROSS-REFERENCE TO RELATED APPLICATION

This application claims priority to U.S. Provisional Patent Application No. 62/694,992, filed on Jul. 7, 2018, which is hereby incorporated by reference in its entirety.

### FIELD

The disclosure relates to a cartridge for storing and diluting concentrated materials and supplying diluted solutions of the concentrate to application equipment.

### BACKGROUND

The microbial contamination of all type of textiles, including fabrics, carpets, curtains, and linens, can contribute to staining of the textiles, unwanted odor, and the spread of disease.

Accordingly, the inventors have identified a need in the art to provide cost effective and convenient products and methods for applying antimicrobial products to textiles and other surfaces in need thereof.

### SUMMARY

In one aspect of the disclosure, an example cartridge is disclosed. The cartridge comprises: (i) a concentrate chamber that stores a concentrate solution and comprising an outlet, (ii) a dilution chamber comprising an inlet for receiving the concentrate solution from the concentrate chamber, and outlet for dispensing a diluted solution, and (iii) a mechanical portion comprising one or more pumps for delivering the concentrate solution to the dilution chamber and for dispensing the diluted solution.

In various aspects of the disclosure, at least one of the concentrate chamber and the dilution chamber are replaceable and/or refillable. In addition, the dilution chamber may include an inlet for a liquid diluent and the one or more pumps may deliver the liquid diluent to the dilution chamber.

In a further aspect of the disclosure, the solution includes a metallic ion, such as for example a silver ion or a copper ion.

In another aspect, the disclosure includes a housing confining the dilution chamber, the concentrate chamber and the mechanical portion. The cartridge may also include the mechanical chamber confined in the housing for containing the mechanical portion.

Still further, the disclosure provides a system for delivering the diluted solution comprising. The system can include a cartridge of the disclosure and a dispensing apparatus. The systems may further include an inlet for receiving the diluted solution from the dilution chamber, and (b) a dispenser that delivering diluted solution.

In various aspects of the system of the disclosure, the system may include a supply of a liquid diluent and a supply outlet, and the dilution chamber may include an inlet for the diluted solution from the supply outlet.

Still further, the disclosure describes a cartridge including an RFID reader for matching the cartridge with the apparatus.

### BRIEF DESCRIPTION OF THE DRAWINGS

FIG. 1A is an example cartridge in a first state.

FIG. 1B is an example cartridge in a second state.

FIG. 1C is an example cartridge in a third state.

FIG. 1D is an example cartridge in a fourth state.

FIG. 1E is an example cartridge in a fifth state.

FIG. 2A is a schematic representation of an integrated cartridge.

FIG. 2B is the example of the integrated cartridge of FIG. 2A.

### DESCRIPTION

The disclosure is directed to cartridges and systems for delivering dilute solutions of cleaning, deodorizing, antimicrobial, antistatic solutions and other useful solutions to textile surfaces. The surfaces include fabrics used in all applications and textile coverings such as carpets, draperies, etc. The systems may include dispensing apparatuses such as a commercial or home-use textile washing machine, or may include surface cleaning devices such as a vacuum cleaner. The cartridges provide a supply of dilute solutions to the apparatuses for delivery to the textile surfaces, and may do so in a number of ways.

In one aspect, the disclosure is directed to a multi-chamber cartridge for storing a supply of a concentrate solution that is diluted and delivered to textile surfaces by a dispensing apparatus. The cartridge also includes a dilution chamber for diluting the solution with a liquid diluent, such as water. A mechanical portion of the cartridge provides the appropriate pumps, controllers, and fluid communication between a concentrate chamber and the dilution chamber. The mechanical portion may also deliver the dilute solution from the dilution chamber to the apparatus.

One or both of the concentrate chamber and the dilution chamber may be replaceable or refillable. For instance, the concentrate chamber may include a leak-proof quick connection (e.g., a “single-click” installation) that provides fluid communication between the chamber and the mechanical portion or dilution chamber of the cartridge. The quick connection allows for consumer replacement that avoids consumer contact with the liquid concentrate. In some aspects, the dilution chamber may be refillable by the consumer, manufacturer or other supplier. Similarly, a diluent container may be replaced and refilled to provide a supply of the appropriate diluent. In addition, the diluent container may be supplied with diluent by the dispensing apparatus. The dilution chamber, the concentrate chamber and the mechanical portion each include the appropriate inlets and outlets for supplying liquid diluent and liquid concentrate to the diluent container, and for supplying the diluted liquid to the dispensing apparatus (e.g., washing machine or vacuum cleaner). The mechanical portion may be integrated throughout the cartridge or may be confined in a mechanical chamber that is in fluid communication, but separate and distinct, from the other chambers.

The diluent may be water or another liquid appropriate for diluting the concentrate solution. In some instances, the concentrate solution includes a metallic ion, such as silver ion or copper ion, which are known for their deodorizing, antimicrobial and antistatic properties when applied to textiles. In addition, the concentrate solution may contain cleaning or deodorizing compounds appropriate for the textiles to which they are applied. The cartridge may include a housing, for example a rigid protective housing for confining the dilution chamber, the concentrate chamber and the mechanical portion or chamber.

In another aspect, the disclosure is directed to a system for delivering the diluted solution. The system includes a dispensing apparatus that has an inlet for receiving the diluted solution from the cartridge and a dispenser that delivers diluted solution to the textile surface. For instance, a washing machine will include a wash basin for applying the diluted solution to textiles in the basin, usually combined with a level of wash or rinse water in the basin. A vacuum cleaner may include a spraying mechanism, a vaporizer or other mechanism that can apply the dilute solution to the textile surfaces.

The dispensing apparatus may include a supply of the liquid diluent and a supply outlet for supplying the liquid diluent to the dilution chamber of the cartridge. The cartridge may include an inlet for the diluent solution from the supply outlet and provide fluid communication to the dilution chamber. A quick connect fluid connector between the supply outlet and the cartridge inlet can allow consumers to replace the cartridge without contacting the solution.

The dispensing apparatus may also provide for a low voltage power supply and connector for supplying power to the cartridge through the appropriate connector on the cartridge.

In some aspects, the system may include an identification system to allow the correct matching of the cartridge to the dispensing apparatus to prevent consumer mistake and avoid counterfeiting. For instance, an RFID reader and an appropriate display may be used for matching a cartridge with the apparatus and warning the consumer of the use of the incorrect cartridge.

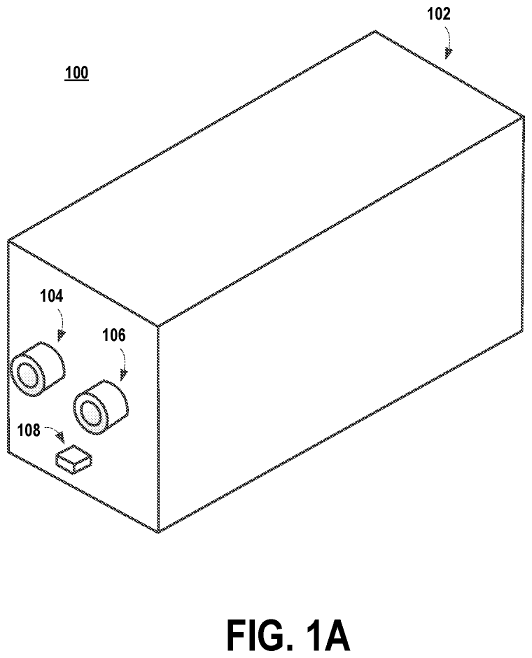

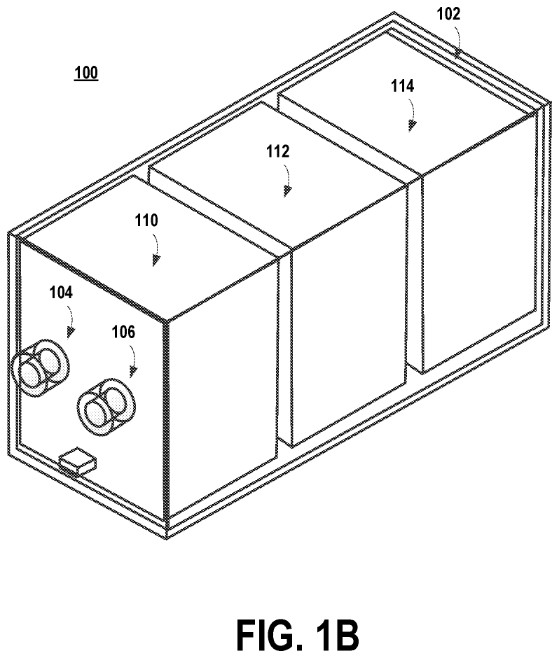

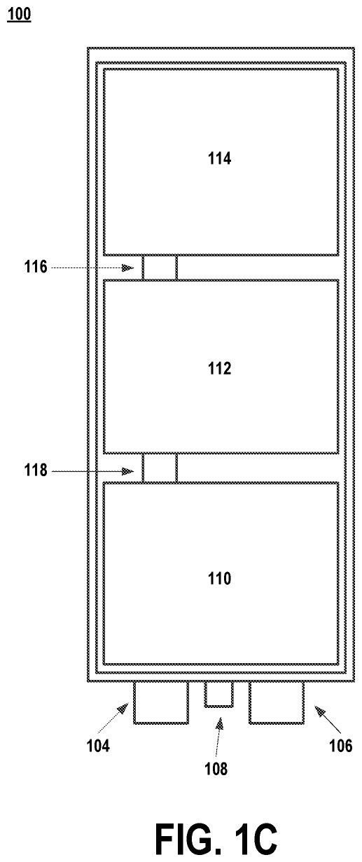

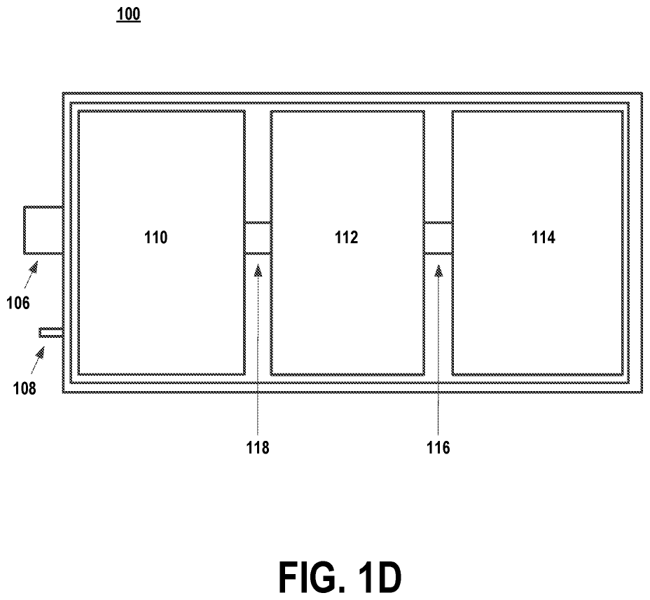

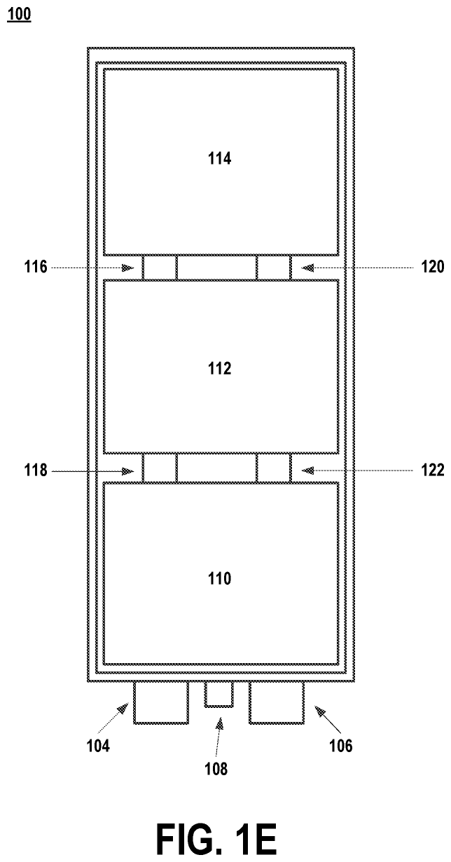

FIGS. 1A-E provide a schematic diagram of an example cartridge according to the disclosure. The features described herein can involve environments, operations, and functionalities that are configured or formatted differently, include additional or fewer components, include different types of components, and relate to one another in different ways. A cartridge 100 provides a liquid diluent (e.g., water), mixes the liquid diluent with a concentrate solution, and dispenses a diluted solution comprising, at least, the liquid diluent and the concentrate solution.

In an example embodiment, as illustrated in FIG. 1A, the cartridge 100 may be made up of a housing 102, fluid connections, including an inlet 104 for receiving liquid diluent, an outlet 106 for dispensing diluted solution, and a power connection 108.

In this embodiment, the housing 102, the inlet 104, and the outlet 106 may be made of thermally-insulative and waterproof materials such as plastic, rubber, a combination of the two, but it should be understood that any of these components may be made of other materials. In a further aspect, the materials used to form any of these components may also be fortified or improved through one or more processes (e.g., chemical treatment).

In this embodiment, the inlet 104 and/or the outlet 106 may be leak-proof quick connections that provide fluid communication between cartridge 100 and an apparatus with which the cartridge is integrated. In a further aspect, because inlet 104 and/or outlet 106 can encompass such quick connections, a consumer may replace or exchange cartridge 100 and avoid contact with any liquid concentrate solution inside the cartridge. These quick connections may provide easy installation for the consumer, thereby obviating any need for professional installment.

Furthermore, the housing 102, the inlet 104, and/or the outlet 106 may also be modified to interact with a variety of connection types and configurations (e.g., washing machine, washing machine drawers, dryers, vacuums, spraying devices, vacuums, refrigerators, humidifiers, steamers, etc.), and the shape and/or dimensions of any component of cartridge 100 can be modified to fit in or integrate with any device.

In one example, the power connection 108 may be configured to integrate with a specific brand or type device with a known power rating (e.g., a vacuum cleaner running on 110 Volts). Alternatively, the power connection 108 may be configured to operate using low power. For example, a dispensing apparatus to which the cartridge 100 is installed may allow any received power be modified to a low power signal (e.g., a step-down transformer). In other examples, however, the use of power connection 108 may not be necessary (e.g., if some or all of the mechanical portion does not require power to operate).

FIG. 1B is an alternate, expanded, rendering of the components of the cartridge of depicted in FIG. 1A. Specifically, in FIG. 1B, the cartridge 100 contains a dilution chamber 110 connected to fluid connections of the inlet 104 and the outlet 106. The dilution chamber 110 is also connected via one or more fluid connections to a mechanical chamber 112, and the mechanical chamber 112 is connected to a concentrate chamber 114 via one or more fluid connections as well. In this embodiment, the dilution chamber 110, the mechanical chamber 112, and the concentrate chamber 114 are contained in a housing 102, and are depicted in a particular configuration. FIG. 1B, however, depicts only one example configuration, and other configurations are possible (and may even be preferable). For example, in another example embodiment, concentrate chamber 114 may be directly connected to dilution chamber 110 via one or more fluid connections, and dilution chamber 110 may be directly connected to mechanical chamber 112.

Turning back to FIG. 1B, in the example embodiment illustrated by FIG. 1B, a solid-walled structure is shown with the housing 102, and fluid connections for receiving liquid diluent, the inlet 104, for dispensing diluted solution, the outlet 106, and a power connection 108. Unlike FIG. 1A, FIG. 1B also shows a dilution chamber 110, a mechanical chamber 112, and a concentrate chamber 114 contained and confined in the housing 102.

In this embodiment, liquid diluent may enter the dilution chamber 110 via the inlet 104. In another embodiment, the dilution chamber 110 may already contain a liquid diluent (e.g., water or other liquids appropriate for diluting concentrated solutions), which may be further supplemented by the liquid diluent or one or more alternative liquid diluents via the inlet 104. In yet another aspect, the dilution chamber 110 may be replaced and/or refilled to provide an adequate supply of one or more appropriate diluents.

The liquid diluent contained in the dilution chamber 110 may be supplemented by a concentrate solution contained in the concentrate chamber 114. The concentrate solution in the concentrate chamber 114 may include a solution containing a metallic ion, such as silver ion or copper ion, among other possibilities. In addition, the concentrate solution in the concentrate chamber 114 may contain cleaning or deodorizing compounds appropriate for the textiles to which they are applied. The concentrate chamber 114 may be replaced and/or refilled to provide an adequate supply of the appropriate concentrate solution.

Furthermore, the concentrate solution in the concentrate chamber 114 may be mixed with the liquid diluent in the dilution chamber 110 by using pumps and controllers (among other potential components) residing in the mechanical portion of the cartridge. For example, the liquid diluent in the dilution chamber 110 may be supplemented by concentrate solution contained in the concentrate chamber 114 by or more electrical pumps configured to provide fluid communication throughout the cartridge 100 and may be contained in a mechanical portion such as a mechanical chamber 112. In a further aspect, the liquid diluent in the dilution chamber 110 may be supplemented by concentrate solution contained in the concentrate chamber 114 based on one or more events. For example, the concentrate solution may be added to the liquid diluent in response to a particular event (e.g, at the onset of a new wash cycle in a washing machine, in response to determining that the ion concentration level of the diluted solution is lower than expected), periodically, or both, among other possibilities.

Additionally, the mechanical portion (and any components contained therein or in communication therewith) may be integrated throughout the cartridge 100 or confined in mechanical chamber 112, or some combination of the two. For example, as described above, the mechanical portion may be integrated between the dilution chamber 110 and the concentrate chamber 114 to facilitate fluid communication between the two. Additionally or alternatively, the mechanical portion may be integrated after the dilution chamber to facilitate the same, as well as other functionalities of the cartridge (e.g., dispensing the diluted solution).

In another example embodiment, the dilution chamber 110 and the concentrate chamber 114 may be in direct fluid communication with each other and the mechanical portion may be integrated after the dilution chamber 110. The mechanical portion can also still be used to facilitate concentrate mixing in the dilution chamber 110, and may do so in a number of ways (e.g., by using a mechanical and/or electromechanical mixing component extending into the dilution chamber, by creating pressure and/or fluid volatility in the dilution chamber, etc.).

Additionally or alternatively, the mechanical portion can also be used to dispense the diluted solution from the cartridge at a particular rate. In some examples, the mechanical portion may dispense the diluted solution using any number or configuration of mechanical and/or electromechanical components (e.g., pumps, controllers, hydraulic valves, manual valves, solenoid valves, fluid regulators, and/or any other components used to facilitate regulated fluid communication). Either way, any of these components may be in fluid communication with, but remain separate and distinct from, the dilution chamber 110 and the concentrate chamber 114.

Turning to FIG. 1C, a top-down view of an example embodiment of the components of the device depicted in FIGS. 1A-1B is shown. Again, like FIGS. 1A and 1B, FIG. 1C depicts only one example configuration, and other configurations are possible (e.g., concentrate chamber 114 may be directly connected to dilution chamber 110 via one or more fluid connections, and dilution chamber 110 may be directly connected to mechanical chamber 112.

In FIG. 1C, the cartridge 100 contains a dilution chamber 110 receiving liquid diluent via an inlet 104, which in turn is supplemented by the concentrate solution from a concentrate chamber 114, by passing the concentrate solution by fluid communication from the outlet 116 to the mechanical chamber 112 to the outlet 118 and then to the dilution chamber 110, before (and/or in addition to) dispensing the diluted solution via the outlet 106, all of which may utilize power received via the power connection 108.

In this embodiment, monitoring and providing consistent concentration of the diluted solution may be advantageous and may be accomplished in one or more ways. In one example, the dilution chamber 110 may contain one or more sensors that are configured to determine and/or monitor the concentration of the concentrate solution in the diluted solution of dilution chamber 110.

For example, if the concentration of concentrate solution exceeds the expected or desired concentration level, one or more actions can be taken by the components in cartridge 100. For example, if concentration is too high, the sensors in dilution chamber may cause other components to decrease the amount of concentrate solution incoming from concentrate chamber 114 (e.g., by closing or reducing the fluid communication of the outlets 116 or 118, or by shutting off one or more pumps in the mechanical chamber 112, or by some combination of the two). Additionally or alternatively, the sensors in the dilution chamber 110 may cause other components of the cartridge 100 to increase the amount of liquid diluent in the dilution chamber 110 (e.g., by increasing the fluid communication of the inlet 104), thereby also lowering the concentration of concentrate solution in the dilution chamber 110.

If, on the other hand, the concentration is lower than expected or desired, one or more actions can be taken by the components in the cartridge 100. For example, if concentration is too low, the sensors in dilution chamber may cause other components to increase the amount of concentrate solution incoming from the concentrate chamber 114 (e.g., by further opening or increasing the fluid communication of the outlets 116 or 118, or by increasing the operation of one or more pumps in the mechanical chamber 112, or by some combination of the two). Additionally or alternatively, the sensors in the dilution chamber 110 may cause other components of the cartridge 100 to decrease the amount of liquid diluent in the dilution chamber 110 (e.g., by decreasing or stopping the fluid communication of the inlet 104), thereby also increasing the concentration of concentrate solution in the dilution chamber 110.

In any event, the dilution chamber 110, the mechanical chamber 112, the concentrate chamber 114, each include appropriate inlets and outlets for supplying liquid diluent and liquid concentrate solution to the diluent container via controlled and intelligent fluid communication, as well as for supplying the final diluted solution to a dispensing apparatus (e.g., washing machine or vacuum cleaner) via the outlet 106.

Additionally, the cartridge 100 may include a component for allowing the device with which it will be integrated to allow the correct matching of the cartridge to the dispensing apparatus to prevent consumer mistake and avoid counterfeiting. For instance, an RFID reader and an appropriate display may be used for matching the cartridge 100 with the apparatus and warning the consumer of the use of the incorrect cartridge (e.g., incorrect brand, incorrect concentrate solution, counterfeit products, etc.). This (or similar) technology may also be used to allow the cartridge 100 to communicate in other ways with the dispensing apparatus.

For example, the cartridge 100 may be enabled with one or more technologies to allow wired communication (e.g., USB, Mini-USB, and/or Lightning connections) and/or wireless communication (e.g., via Bluetooth, Near Field Communication, WI-FI, and/or other wireless connections), among other possibilities.

FIGS. 1D and 1E, show a side view and a top view of the example embodiments shown in depicted in FIGS. 1A-1C. Specifically, in FIG. 1D, the cartridge 100 contains a the dilution chamber 110 containing liquid diluent, which in turn is supplemented by concentrate solution from the concentrate chamber 114, by passing the concentrate solution via fluid communication from the outlet 116 to the mechanical chamber 112 to the outlet 118 and then to the dilution chamber 110, before (and/or in addition to) dispensing the diluted solution via the outlet 106, all of which may utilize power received from the power connection 108.

Unlike FIG. 1C, in FIG. 1E. the cartridge 100 also contains additional mechanisms for passing solution from the concentrate chamber 114 via fluid communication from the outlet 120 to the mechanical chamber 112 to the outlet 122 and then to dilution chamber 110, before (and/or in addition to) dispensing the diluted solution via the outlet 106, all of which also utilizes power received via power connection 108.

In this embodiment, more detailed consistent concentration of the diluted solution may be monitored and provided as the one or more sensors that are configured to determine and/or monitor the concentration of the concentrate solution. One or more pumps (or different or equivalent devices) in the mechanical chamber 112 can ensure the correct concentration of concentrate solution in the diluted solution to be dispensed via the outlet 106.

For example, if the concentration is lower than expected or desired, one or more actions can be taken by the components in cartridge 100. For example, if concentration is too low, the sensors in the dilution chamber 110 may cause other components to dynamically increase the amount of concentrate solution incoming from the concentrate chamber 114 (e.g., by further opening or increasing the fluid communication of the outlets 116, 118, 120, 122, or by increasing the operation of one or more pumps in the mechanical chamber 112, or by some dynamic combination of the two).

In any event, in this embodiment as well, the dilution chamber 110, the mechanical chamber 112, and the concentrate chamber 114, each include appropriate inlets and outlets for supplying liquid diluent and liquid concentrate solution to the dilution chamber via controlled and intelligent fluid communication, as well as for supplying the final diluted solution to a dispensing apparatus (e.g., washing machine or vacuum cleaner) via the outlet 106.

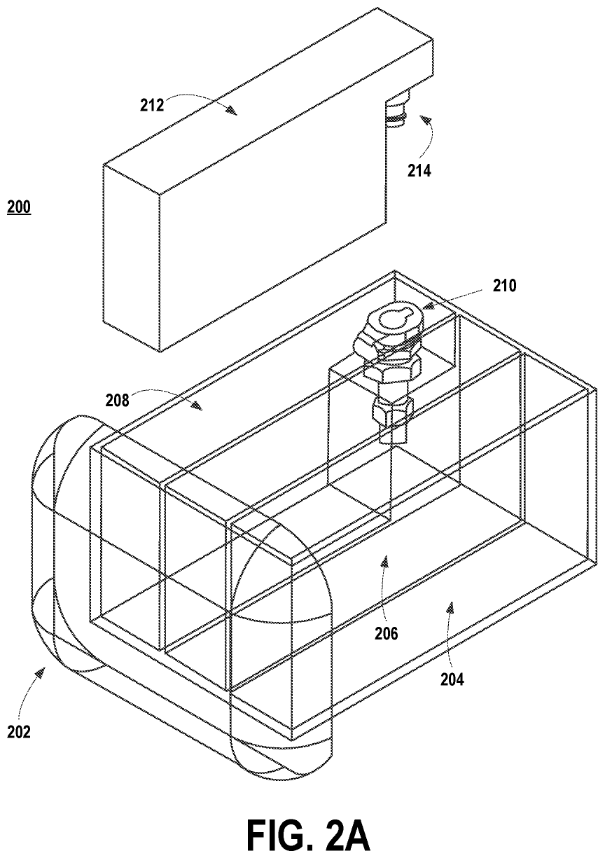

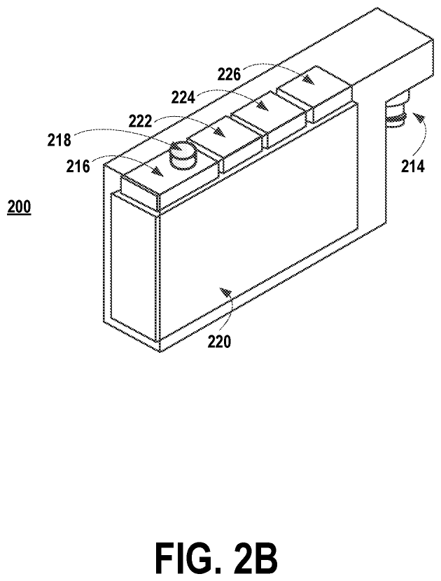

FIG. 2A shows a schematic representation of an integrated cartridge 200 for receiving a liquid diluent (e.g., water), mixing the liquid diluent with a concentrate solution, and dispensing a diluted solution comprising, at least, the liquid diluent and the concentrate solution. In an example embodiment, as illustrated in FIG. 2A, the cartridge 200 may contain a housing 202 made of thermally-insulative and waterproof materials such as plastic, rubber, or a combination of the two.

In this embodiment, the cartridge 200 may also contain one or more the chambers 204 and 206 for containing and mixing liquid diluent with concentrate solution from a concentrate chamber 208. In this embodiment, the chambers 204 and 206 may also be in connection with each other and the concentrate chamber 114 via one or more fluid connections. All of the chambers 204 and 206, and the concentrate chamber 208 may also be contained in the housing 202.

Further, the concentrate chamber 208 may be supplemented (or supplied entirely) with concentrate solution via the integration of the concentrate cartridge 212, in connection with the housing 202 (and the chambers 204 and 206, and concentrate chamber 208), by the coupling of the inlet 210 and the outlet 214. In the illustrated embodiment, the inlet 210 and the outlet 214 may be made of thermally-insulative and waterproof materials such as plastic, rubber, or a combination of the two, like the housing 202, but may also be made of a variety of other materials proving quick, simple, and secure connection and fluid communication.

In a further aspect, because the inlet 210 and/or outlet the 214 can encompass quick connections, a consumer may replace or exchange the concentrate cartridge 212 and avoid contact with any liquid concentrate inside the cartridge. Furthermore, these quick connections may provide easy installation for the consumer, thereby obviating any need for professional installment, and may be replaced and/or refilled to provide an adequate supply of the appropriate concentrate solution.

Either way, the liquid diluent contained in the chambers 204 and 206 may be supplemented by a concentration solution contained in the concentrate chamber 208 and the concentrate cartridge 212, which may include a solution containing a metallic ion.

Furthermore, the concentrate solution in the concentrate chamber 114 may be mixed the liquid diluent in the dilution chamber 110 by using a number of electrical and mechanical devices and based on a number of factors. In a further aspect, the concentrate cartridge 212 may contain a variety of components, configurations, and functionality, ranging from complex (e.g., like the cartridge illustrated and discussed in the context of FIG. 1B), or more straightforward (e.g., only contain the concentrate solution to be distributed via the outlet 214).

In this embodiment as well, the chambers 204 and 206, the concentrate chamber 208, and the concentrate cartridge 212 each include appropriate inlets and outlets for supplying liquid diluent and liquid concentrate solution to the one or more dilution chambers via controlled and intelligent fluid communication, as well as for supplying the final diluted solution to a dispensing apparatus (e.g., washing machine or vacuum cleaner).

FIG. 2B shows a schematic representation of integrated cartridge 200, but in a second state. Specifically, in an example embodiment, as illustrated in FIG. 2B, the cartridge 200 may also contain a concentrate chamber 216 with solution mix button 218 and in direct fluid communication with dilution chamber 220, all of which may be made of thermally-insulative and waterproof materials such as plastic, rubber, or a combination of the two.

In this embodiment, the cartridge 200 may also contain one or more mechanical portions contained in chambers 222, 224, and 226. In one example, chamber 222 may contain one or more communication mechanisms (e.g., one or more technologies allowing wireless communication with the dispensing apparatus via Bluetooth, Near Field Communication, WI-FI, and/or other wireless connections).

In a further aspect, chamber 224 may contain any number or configuration of mechanical and/or electromechanical components used to facilitate fluid communication between the concentrate chamber 216 and dilution chamber 220, out of the dilution chamber 220 and into the dispensing mechanism, or both (e.g., via one or more pumps, controllers, hydraulic valves, manual valves, solenoid valves, fluid regulators, and/or any other components used to facilitate regulated fluid communication).

In a further aspect, chamber 226 may contain one or more computing devices enabled to control one or more components of cartridge 200. For example, chamber 226 can contain a computing device that can perform various acts and/or functions via one or more components, such one or more processors and data storage such as a non-transitory computer-readable storage medium, having stored thereon program instructions (e.g., compiled or non-compiled program logic and/or machine code) that, when executed by the one or processors, cause the computing device to perform one or more acts and/or functions, such as those described in this disclosure.

In yet a further aspect, some or all of the mechanical portions contained in contained in chambers 222, 224, and 226 may be configured to integrate with a specific brand or type device with a known power rating (e.g., a washing machine running on 110 Volts) or may be configured to operate using a power source integrated into the cartridge (e.g., via one or more rechargeable batteries). And again, FIG. 2B, however, depicts only one example configuration, and other configurations are possible (and may even be preferable).

For example, although, like FIG. 2A, FIG. 2B shows outlet 214 as an example mechanism for facilitating fluid exchange between cartridge 200 and the dispensing apparatus with which cartridge 200 communicates. In another example embodiment, however, cartridge 200 may exchange fluid and/or dispense diluted solution via inlets and/or outlets are different than the quick connect depicted in FIGS. 2A and 2B, and/or in response to different events than discussed above.

For example, cartridge 200 may be a self-contained and self-powered unit that operates in a chamber (e.g., concentrate chamber 208) and dispenses diluted solution based on one or more events. For example, cartridge 200 may detect that the chamber is filling with water and, in response to determining the presence of water, the cartridge 200 can mix and administer a diluted solution into the chamber. In another example, the cartridge could mix and administer a diluted solution based on receiving one or more commands from the device in which the cartridge is operating (e.g., based on receiving a command, via Bluetooth, that a new wash cycle is beginning).

The singular forms of the articles “a,” “an,” and “the” include plural references unless the context clearly indicates otherwise. For example, the term “a compound” or “at least one compound” can include a plurality of compounds, including mixtures thereof.

Various aspects and embodiments have been disclosed herein, but other aspects and embodiments will certainly be apparent to those skilled in the art. Additionally, the various aspects and embodiments disclosed herein are provided for explanatory purposes and are not intended to be limiting, with the true scope being indicated by the following claims.

1-14. (canceled)

15. A cartridge comprising:
a concentrate chamber that stores a concentrate solution and comprising an outlet,
a dilution chamber comprising an inlet for receiving the concentrate solution from the concentrate chamber, and outlet for dispensing a diluted solution, and
a mechanical portion comprising one or more pumps for delivering the concentrate solution to the dilution chamber and for dispensing the diluted solution.

16. The cartridge of claim 15, wherein at least one of the concentrate chamber and the dilution chamber are replaceable.

17. The cartridge of claim 15, wherein at least one of the concentrate chamber and the dilution chamber are refillable.

18. The cartridge of claim 15, wherein the dilution chamber comprises an inlet for a liquid diluent.

19. The cartridge of claim 18, wherein the one or more pumps delivers the liquid diluent to the dilution chamber.

20. The cartridge of claim 15, wherein the concentrate solution comprises a metallic ion.

21. The cartridge of claim 20, wherein the metallic ion comprises a silver ion.

22. The cartridge of claim 20, wherein the metallic ion comprises a copper ion.

23. The cartridge of claim 15, further comprising a housing confining the dilution chamber, the concentrate chamber, and the mechanical portion.

24. The cartridge of claim 23, wherein the housing comprises a thermally-insulative and waterproof material.

25. The cartridge of claim 23, further comprising a mechanical chamber confined in the housing for containing the mechanical portion, wherein the mechanical chamber is in fluid communication with the dilution chamber and the concentrate chamber, and wherein the mechanical chamber is separate and distinct from the dilution chamber and the concentrate chamber.

26. The cartridge of claim 23, further comprising an RFID reader.

27. A system for delivering the diluted solution, the system comprising:
the cartridge of claim 15; and
a dispensing apparatus comprising (a) an inlet for receiving the diluted solution from the dilution chamber, and (b) a dispenser that delivering diluted solution.

28. The system of claim 27, wherein the dispensing apparatus comprises a supply of a liquid diluent and a supply outlet, and the dilution chamber comprises an inlet for the diluted solution from the supply outlet.

29. The system of claim 27, wherein the cartridge comprises an RFID reader for matching the cartridge with the apparatus.

30. The system of claim 27, wherein the dispensing apparatus delivers the dilute solution to textiles.

31. The system of claim 27, wherein the dispensing apparatus delivers the dilute solution to a washing machine basin.

32. The system of claim 27, further comprising an identification system, wherein the identification system comprises an RFID reader for matching the cartridge with the apparatus.

33. The system of claim 32, wherein the identification system further comprises a display of a warning that the cartridge does not match the apparatus.

34. The system of claim 33, wherein the display of a warning the cartridge does not match the apparatus is based on the identification system detecting, via the RFID reader, that the cartridge does not match the apparatus.
